(19) United States
(12) Patent Application Publication
(10) Pub. No.: US 2023/0404692 A1
(43) Pub. Date: Dec. 21, 2023

(54) COST EFFECTIVE ROBOTIC SYSTEM ARCHITECTURE

(71) Applicant: Mazor Robotics Ltd., Caesarea (IL)

(72) Inventors: Yonatan Ushpizin, Glil Yam (IL); Eliyahu Zehavi, Tel Aviv (IL); Avraham Turgeman, Beer Yaakov (IL)

(21) Appl. No.: 17/845,032

(22) Filed: Jun. 21, 2022

(51) Int. Cl. A61B 34/37

(52) U.S. Cl. CPC A61B 34/37; A61B 2017/00477

(57) ABSTRACT

Systems, techniques, and embodiments are provided herein that are configured to interchangeably couple any surgical tool of a plurality of surgical tools to a robotic arm. For example, a robotic surgical system is provided that includes an interface unit disposed at a distal end of the robotic arm and coupled to the robotic arm, where the interface unit includes a set of components configured to provide the coupling. In some embodiments, the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm. Additionally, each surgical tool of the plurality of surgical tools may comprise an identification feature for the interface unit and/or a processor to identify which surgical tool is being coupled to the robotic arm.

### BACKGROUND

The present disclosure is generally directed to surgical systems, and relates more particularly to robotic surgical devices.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A robotic surgical system, comprising: a robotic arm comprising a proximal end and a distal end; a plurality of surgical tools; an interface unit disposed at the distal end of the robotic arm and coupled to the robotic arm, wherein the interface unit comprises a set of components configured to interchangeably couple any surgical tool of the plurality of surgical tools to the robotic arm; a processor coupled with the robotic arm and the interface unit; and a memory coupled with and readable by the processor and storing data for processing by the processor, the data, when processed, causes the processor to: determine that a first surgical tool of the plurality of surgical tools is coupled to the interface unit via the set of components; and transmit instructions to operate the first surgical tool based at least in part on determining that the first surgical tool is coupled to the interface unit.

Any of the aspects herein, wherein the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm.

Any of the aspects herein, wherein the memory stores further data for processing by the processor that, when processed, causes the processor to: determine a first set of operations corresponding to the first surgical tool based at least in part on determining that the first surgical tool is coupled to the interface unit, wherein the instructions to operate the first surgical tool are based at least in part on the first set of operations.

Any of the aspects herein, wherein the memory stores further data for processing by the processor that, when processed, causes the processor to: determine that a second surgical tool of the plurality of surgical tools is coupled to the interface unit via the set of components; and transmit instructions to operate the second surgical tool based at least in part on determining that the second surgical tool is coupled to the interface unit.

Any of the aspects herein, wherein the memory stores further data for processing by the processor that, when processed, causes the processor to: determine a second set of operations corresponding to the second surgical tool based at least in part on determining that the second surgical tool is coupled to the interface unit, wherein the instructions to operate the second surgical tool are based at least in part on the second set of operations.

Any of the aspects herein, wherein the second set of operations is different than the first set of operations.

Any of the aspects herein, wherein the second surgical tool replaces the first surgical tool being coupled to the interface unit via the set of components.

Any of the aspects herein, wherein each surgical tool of the plurality of surgical tools comprises an identification feature.

Any of the aspects herein, wherein the data stored by the memory that, when processed, causes the processor to determine the first surgical tool of the plurality of surgical tools is coupled to the interface unit via the set of components causes the processor to: identify, when the first surgical tool is coupled to the interface unit, the first surgical tool based at least in part on a first identification feature of the first surgical tool.

Any of the aspects herein, wherein the identification feature comprises a quick response (QR) code, a serial number, a barcode, an inner memory stored inside each surgical tool, a visual marker, a radio-frequency identification (RFID) system, a Near-Field Communication (NFC) connection, inner switching of an input/output (I/O) system, or a combination thereof.

A robotic surgical system, comprising: a robotic arm comprising a proximal end and a distal end; a plurality of surgical tools; an interface unit disposed at the distal end of the robotic arm and coupled to the robotic arm, wherein the interface unit comprises a set of components configured to interchangeably couple any surgical tool of the plurality of surgical tools to the robotic arm.

Any of the aspects herein, wherein the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm.

Any of the aspects herein, wherein each surgical tool of the plurality of surgical tool corresponds to a specific set of operations.

Any of the aspects herein, wherein each surgical tool of the plurality of surgical tools comprises an identification feature.

Any of the aspects herein, wherein the identification feature comprises a QR code, a serial number, a barcode, an inner memory stored inside each surgical tool, a visual marker, an RFID system, an NFC connection, inner switching of an I/O system, or a combination thereof.

A robotic surgical system, comprising: a robotic arm comprising a proximal end and a distal end; a plurality of surgical tools; an interface unit disposed at the distal end of the robotic arm and coupled to the robotic arm, comprising: a body; a tool receiving element; and a set of components configured to interchangeably couple any surgical tool of the plurality of surgical tools to the robotic arm.

Any of the aspects herein, wherein the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm.

Any of the aspects herein, wherein each surgical tool of the plurality of surgical tool corresponds to a specific set of operations.

Any of the aspects herein, wherein each surgical tool of the plurality of surgical tools comprises an identification feature.

Any of the aspects herein, wherein the identification feature comprises a QR code, a serial number, a barcode, an inner memory stored inside each surgical tool, a visual marker, an RFID system, an NFC connection, inner switching of an I/O system, or a combination thereof.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a perspective diagram of a robotic surgical system according to at least one embodiment of the present disclosure;

FIG. 2B is a perspective diagram of a robotic surgical system according to at least one embodiment of the present disclosure;

FIG. 3A is a perspective diagram of a robotic surgical system according to at least one embodiment of the present disclosure;

FIG. 3B is a perspective diagram of a robotic surgical system according to at least one embodiment of the present disclosure;

FIG. 4 is a flowchart according to at least one embodiment of the present disclosure; and

FIG. 5 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., random-access memory (RAM), read-only memory (ROM), electrically erasable programmable ROM (EEPROM), flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Surgical robots may assist a surgeon or other medical provider in carrying out a surgical procedure or may complete one or more surgical procedures autonomously. Providing controllable linked articulating members allows a surgical robot to reach areas of a patient anatomy during various medical procedures. In some examples, the surgical robots may be part of robotic surgical systems, surgical robotic systems, surgical systems, robotic systems, etc. For example, the robotic surgical systems may at least include a robot arm as a controllable linked articulating member designed to mimic and/or assist in movements and operations normally performed by a surgeon. Additionally, the robotic surgical systems and robot arm(s) may include and/or be designed to work with multiple different tools for performing surgical procedures, where the tools have been or need to be specifically designed and manufactured for use in the robotic surgical systems. For example, the different tools may include a power drill, a powered screwdriver, a power burr, etc. Following a standard design of such tools, each tool may require separate designs for control and electronic components (e.g., designed and built into each tool). The separate designs for control and electronic components for each tool may increase design and manufacture costs associated with each tool.

As described herein, a robotic surgical system is provided that includes separating electronics and control components from tools used as part of the robotic surgical system and placing a single integrated unit (e.g., interface unit) at the tip of a robot arm of the robotic surgical system. The integrated unit may remain connected to the robot arm of the robotic surgical system, such that the tools can be replaced quickly and easily for different operations performed by the robotic surgical system as part of a surgical procedure. The integrated unit may be designed to support a wide variety of tools and can adjust an operating method to fit each one of the tools. Accordingly, the tools used in the robotic surgical system may have simpler designs (e.g., without specific control and electronic component designs for each tool) that are cheaper to manufacture with minimal electronics (e.g., as compared to tools designed to include the specific control and electronic component designs). Additionally, with fewer components and the minimal electronics, a sterilization process for the tools and/or the robotic surgical system may be simplified. In some examples, the integrated unit provided herein may use identification features associated with each of the tools to identify which tool is coupled or connected to the integrated unit (e.g., a quick response (QR) code, serial number (S/N), a barcode, inner memory inside the tools, a visual marker, a radio-frequency identification (RFID) system, a Near-Field Communication (NFC) connection, inner switching of an input/output (I/O) system, etc.), where an operating method for the tool is determined based on identifying which tool is coupled or connected to the integrated unit.

Embodiments of the present disclosure provide technical solutions to one or more of the problems of (1) individually designing electronic and control components for using respective tools in a robotic surgical system, (2) complicated sterilization processes, and (3) expensive tools for use in robotic surgical systems. For example, embodiments of the present disclosure may provide simplified tools, cost reductions, and a reduction in the number of components in a given robotic surgical system by developing an integrated unit (e.g., interface unit) that can accommodate any surgical tool to be coupled to the robotic surgical system.

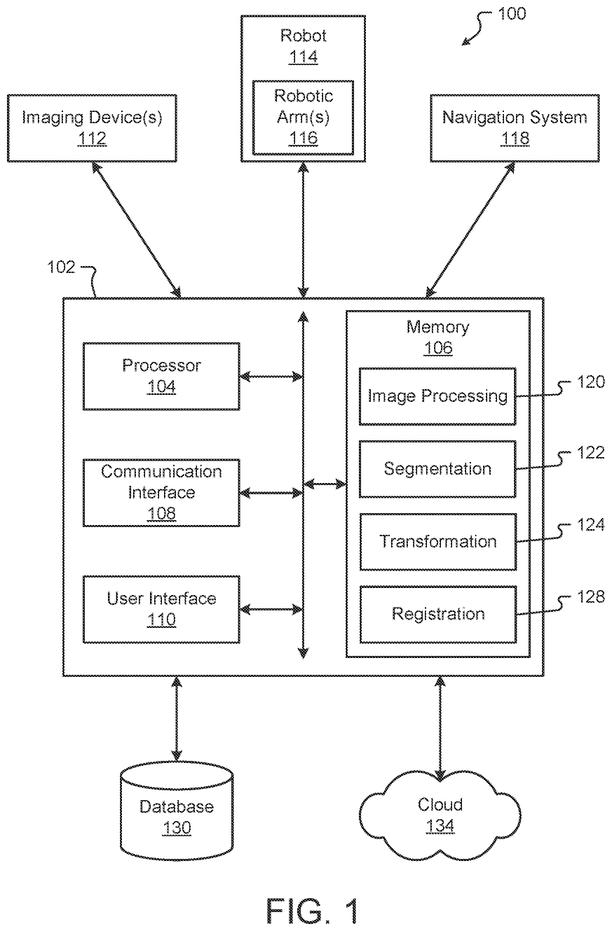

Turning first to FIG. 1, a block diagram of a system 100 according to at least one embodiment of the present disclosure is shown. The system 100 may be used to perform a surgical procedure with surgical tools that can be interchangeably coupled to the system 100 via an interface unit described herein. Additionally or alternatively, the system 100 may be used to control, pose, and/or otherwise manipulate a surgical mount system, a surgical arm, and/or surgical tools attached thereto and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, dynamic RAM (DRAM), synchronous DRAM (SDRAM), other solid-state memory, any memory described herein, or any other tangible, non-transitory memory for storing computer-readable data and/or instructions. The memory 106 may store information or data useful for completing, for example, any step of the methods 400 and/or 500 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a Universal Serial Bus (USB) port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

As described herein, the system 100 may enable separating electronics and control components from surgical tools used as part of the system 100 (e.g., to perform surgical procedures) and placing an interface unit (e.g., single integrated unit) at the tip of a robotic arm 116 (e.g., distal end) of the robot 114 that is configured to accommodate different surgical tools. The interface unit may remain connected to the robotic arm 116, such that the surgical tools can be replaced quickly and easily for performing respective operations of the surgical procedure. For example, the integrated unit may be designed to support a wide variety of surgical tools and can adjust an operating method to fit each one of the surgical tools. That is, the interface unit may be configured to provide electrical coupling, communication coupling, mechanical coupling, pneumatic coupling, irrigation coupling, or a combination thereof, for coupling any surgical tool of a given set of surgical tools to the robotic arm 116 to enable the processor 104 and/or robot 114 to control a respective surgical tool during the surgical procedure.

In some examples, the integrated unit may use identification features associated with each of the surgical tools to identify which surgical tool is coupled or connected to the integrated unit (e.g., via a QR code, a S/N, a barcode, an inner memory inside the surgical tools, a visual marker, an RFID system, an NFC connection, inner switching of an I/O system, etc.). Accordingly, the processor 104 may identify which surgical tool is coupled to the interface unit and may then determine a corresponding operating method for the surgical tool based on identifying the surgical tool that is coupled or connected to the integrated unit. Subsequently, the processor 104 (and/or other components of the system 100) may operate or control the coupled surgical tool via the interface unit.

The system 100 or similar systems may be used, for example, to carry out one or more aspects of any of the methods 400 and/or 500 described herein. The system 100 or similar systems may also be used for other purposes.

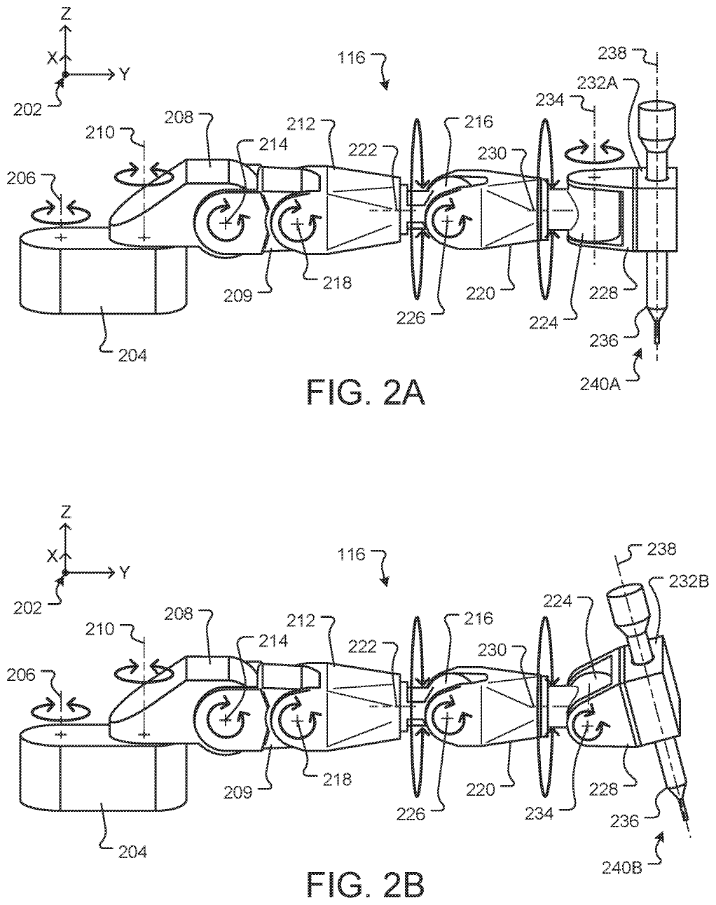

Referring now to FIGS. 2A and 2B, perspective diagrams of a robotic surgical system with different end effector 240A, 240B mount positions are shown in accordance with examples of the present disclosure. More specifically, FIGS. 2A and 2B show the robotic arm 116 of the robot 114 connected to an end effector 240A, 240B holding a surgical tool 236. The surgical tool 236 may correspond to different surgical tools used between operations in a surgical application. For instance, a first surgical tool 236 may include a direction-specific blade that may require a specific rotational alignment and placement in the tool block 232A, 232B, while another surgical tool 236 may include a unidirectional cutting tool that is independent of rotational alignment in the tool block 232A, 232B.

Features of the robot 114 and/or robotic arm 116 may be described in conjunction with a coordinate system 202. The coordinate system 202, as shown in FIGS. 2A and 2B, includes three-dimensions comprising an X-axis, a Y-axis, and a Z-axis. Additionally or alternatively, the coordinate system 202 may be used to define planes (e.g., the XY-plane, the XZ-plane, and the YZ-plane) of the robot 114 and/or robotic arm 116. These planes may be disposed orthogonal, or at 90 degrees, to one another. While the origin of the coordinate system 202 may be placed at any point on or near the components of the robot 114, for the purposes of description, the axes of the coordinate system 202 are always disposed along the same directions from figure to figure, whether the coordinate system 202 is shown or not. In some examples, reference may be made to dimensions, angles, directions, relative positions, and/or movements associated with one or more components of the robot 114 and/or robotic arm 116 with respect to the coordinate system 202. For example, the width of the robotic arm 116 (e.g., running from the side shown in the foreground to the side in the background, into the page) may be defined as a dimension along the X-axis of the coordinate system 202, the height of the robotic arm 116 may be defined as a dimension along the Z-axis of the coordinate system 202, and the length of the robotic arm 116 (e.g., running from a proximal end at the first link 204 to a distal end at the seventh link 224, etc.) may be defined as a dimension along the Y-axis of the coordinate system 202. Additionally or alternatively, the height of the system 100 may be defined as a dimension along the Z-axis of the coordinate system 202, a reach of the robotic arm 116 may be defined as a dimension along the Y-axis of the coordinate system 202, and a working area of the robotic arm 116 may be defined in the XY-plane with reference to the corresponding axes of the coordinate system 202.

The robotic arm 116 may be comprised of a number of links 204, 208, 209, 212, 216, 220, 224 that interconnect with one another at respective axes of rotation 206, 210, 214, 218, 222, 226, 230, 234, or joints. There may be more or fewer links 204, 208, 209, 212, 216, 220, 224 and/or axes of rotation 206, 210, 214, 218, 222, 226, 230, 234 than are shown in FIGS. 2A and 2B. In any event, the robotic arm 116 may have a first link 204 disposed at a proximal end of the robotic arm 116 and an end mount flange 228 disposed furthest from the proximal end at a distal end of the robotic arm 116. The first link 204 may correspond to a base of the robotic arm 116. In some examples, the first link 204 may rotate about first rotation axis 206. A second link 208 may be connected to the first link 204 at a second rotation axis 210, or joint. The second link 208 may rotate about the second rotation axis 210. In one example, the first rotation axis 206 and the second rotation axis 210 may be arranged parallel to one another. For instance, the first rotation axis 206 and the second rotation axis 210 are shown extending along the Z-axis in a direction perpendicular to the XY-plane.

The robotic arm 116 may comprise a third link 209 that is rotationally interconnected to the second link 208 via the third rotation axis 214, or joint. The third rotation axis 214 is shown extending along the X-axis, or perpendicular to the first rotation axis 206 and second rotation axis 210. In this position, when the third link 209 is caused to move (e.g., rotate relative to the second link 208), the third link 209 (and the components of the robotic arm 116 extending from the third link 209) may be caused to move into or out of the XY-plane. The fourth link 212 is shown rotationally interconnected to the third link 209 via the fourth rotation axis 218, or joint. The fourth rotation axis 218 is arranged parallel to the third rotation axis 214. The fourth rotation axis 218 extends along the X-axis allowing rotation of the fourth link 212 into and out of the XY-plane.

In some examples, the robotic arm 116 may comprise one or more wrists 216, 224. The fifth link 216, or wrist, is shown rotationally interconnected to the fourth link 212 via a fifth rotation axis 222, or wrist joint. The fifth rotation axis 222 is shown extending along the Y-axis, which is perpendicular to the X-axis and the Z-axis. During operation of the robot 114, causing the fifth link 216 to rotate about the fifth rotation axis 222 may cause the components of the robotic arm 116 distal the joint at the fifth rotation axis 222 (e.g., the fifth link 216, the sixth link 220, the seventh link 224, the end mount flange 228, and the end effector 240A, 240B, etc.) to rotate about the Y-axis.

The sixth link 220 is rotationally interconnected to the fifth link 216 via the sixth rotation axis 226. The sixth rotation axis 226 extends along the X-axis and provides for rotation of the sixth link 220 relative to the fifth link 216 (e.g., into and out of the XY-plane in the position shown).

The seventh link 224, or wrist, is shown rotationally interconnected to the sixth link 220 via a seventh rotation axis 230, or wrist joint. The seventh rotation axis 230 is shown extending along the Y-axis (e.g., perpendicular to the X-axis and the Z-axis). During operation of the robot 114, causing the seventh link 224 to rotate about the seventh rotation axis 230 may cause the components of the robotic arm 116 distal the joint at the seventh rotation axis 230 (e.g., the end mount flange 228, and the end effector 240A, 240B, etc.) to rotate about the Y-axis.

Located at the distal end of the robotic arm 116, an end mount flange 228 may be rotationally interconnected to the end mount flange 228 via an eighth, or mount flange rotation, axis 234. In FIG. 2A, the seventh link 224 is positioned rotationally about the seventh rotation axis 230 such that the end mount flange 228 is oriented where the mount flange rotation axis 234 is extending along the Z-axis. In FIG. 2B, the seventh link 224 is positioned rotationally about the seventh rotation axis 230 such that the end mount flange 228 is oriented where the mount flange rotation axis 234 is extending along the X-axis. In some examples, at least the seventh link 224 may be rotated about the seventh rotation axis 230 to move between the end mount flange 228 position shown in FIG. 2A and the end mount flange 228 position shown in FIG. 2B, or vice versa. The end mount flange 228 and the mount flange rotation axis 234 may be the last movable (e.g., motor actuated, etc.) link and joint of the robotic arm 116. Moving between these two positions of the end mount flange 228 allows a particular end effector 240A, 240B to be attached and manipulated, or operated, according to a corresponding movement profile (e.g., range and limits) or set of kinematic solutions for the robot 114 (e.g., the robotic arm 116 and the surgical tool 236, etc.).

FIG. 2A shows first movement kinematics for the robotic arm 116 when the first tool block 232A of the first end effector 240A disposes the surgical tool axis 238 parallel to the mount flange rotation axis 234. In the position shown in FIG. 2A, rotation into and/or out of the XY-plane between the seventh link 224 and the first end effector 240A is prevented. This position and arrangement may be ideal for applications (e.g., operations, procedures, etc.) where an end rotational position of the surgical tool 236 may need to be maintained for the robotic arm 116. For example, the surgical tool 236 in the first end effector 240A may correspond to an imaging device that may need to be maintained in a particular nonrotational position relative to a patient during imaging (e.g., where an imaging plane of the surgical tool 236 should be maintained parallel to the XY-plane as other joints of the robotic arm 116 move the distal end closer to or further from the proximal end). In this case, the corresponding arrangement of the surgical tool axis 238 (e.g., parallel to the mount flange rotation axis 234) associated with the first end effector 240A may be preferred. In another example, rotation of the surgical tool 236 into, or out of, the XY-plane may need to be prevented to ensure accuracy of movement along the Y-axis, in the XY-plane, and/or the like. Additionally or alternatively, a distance between a reference plane and an end of the surgical tool 236 (e.g., along the Z-axis) may need to remain constant during operation of the robot 114. In any of these cases, the position and arrangement shown in conjunction with FIG. 2A (e.g., preventing end rotation relative to the XY-plane) may be preferred.

FIG. 2B shows second movement kinematics for the robotic arm 116 when the second tool block 232B of the second end effector 240B disposes the surgical tool axis 238 perpendicular (e.g., at 90 degrees) to the mount flange rotation axis 234. In this alternative position, the end mount flange 228 and second end effector 240B may be allowed to rotate relative to the seventh link 224. Stated another way, in this alternative position, the end mount flange 228 and second end effector 240B may be allowed to rotate into and/or out of the XY-plane (e.g., relative to seventh link 224 at the mount flange rotation axis 234). This position and arrangement may be ideal when a precise rotational movement of the surgical tool 236 at the distal end of the robotic arm 116 is desired. In contrast to the position and arrangement shown in FIG. 2A, where the closest rotation of the first end effector 240A about the X-axis is provided at the sixth rotation axis 226, the position and arrangement of FIG. 2B allows the second end effector 240B to be rotated about the X-axis about the mount flange rotation axis 234. Among other things, this position and arrangement may be used for any application where a movement of the second end effector 240B including an end rotation into and/or out of the XY-plane is desired for the surgical tool 236. Such applications may include directional cutting operations, probing movements, displacement of tissue and organs, and/or other surgical operations.

As described herein, the end mount flange 228, the tool blocks 232, or a different interface unit not explicitly shown in the example of FIGS. 2A and 2B may include a set of components that are configured to interchangeably couple one surgical tool 236 of a plurality of surgical tools to the robotic arm 116 of the robot 114. For example, to perform a surgical procedure, multiple different surgical tools 236 may be used and attached to the robotic arm 116. Rather than having each surgical tool 236 include specific electronic and control components for operating the corresponding surgical tool 236, the set of components may be configured to provide electrical coupling, communication coupling, mechanical coupling, pneumatic coupling, irrigation coupling, or a combination thereof, for coupling a surgical tool 236 to the robot arm 116, such that the robot 114 is capable of controlling the surgical tool 236 regardless of what type of tool the surgical tool 236 is (e.g., drill, screwdriver, blade for performing incisions, burr, blunt tip for displacing tissue, trocar, etc.), what operations are supported or performed by the surgical tool 236, etc. In some examples, the plurality of surgical tools that can be interchangeably coupled to the robot arm 116 (e.g., via the set of components of the end mount flange 228, the tool blocks 232, or the different interface unit) may be specifically manufactured to enable the interchangeable coupling to the robot arm 116.

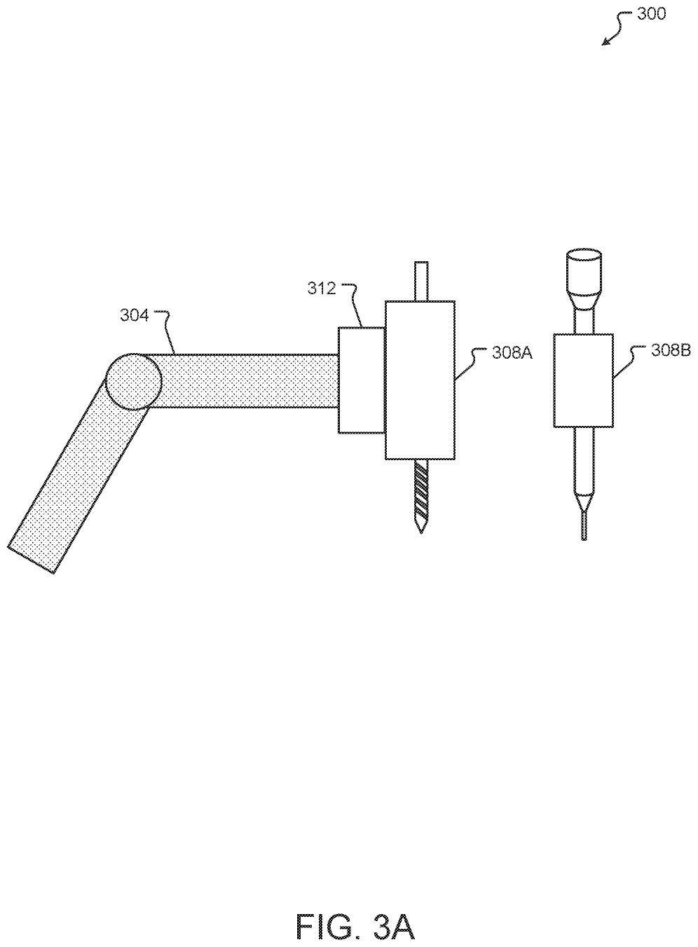

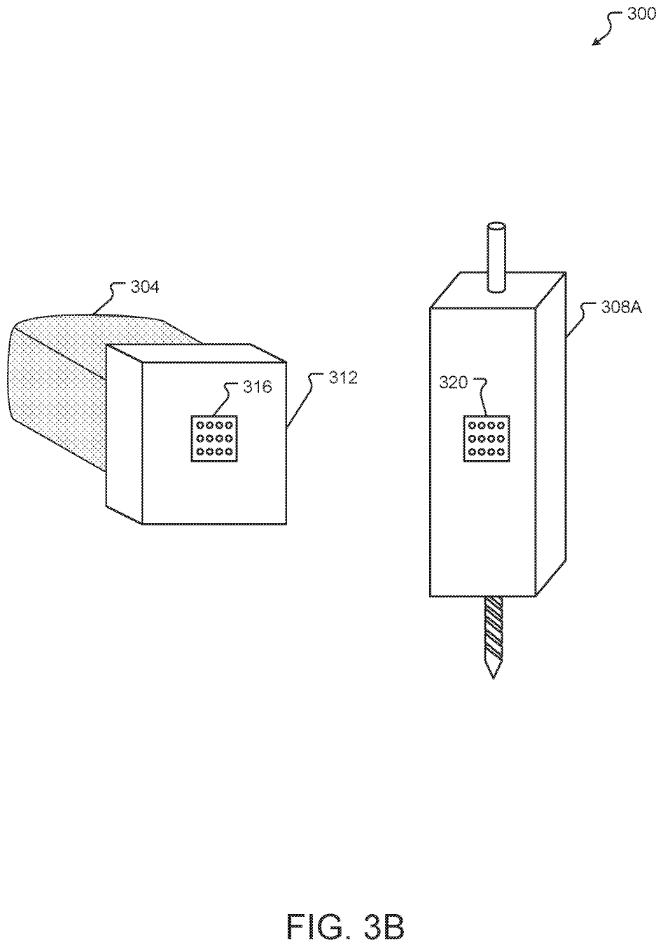

FIGS. 3A and 3B depict perspective views of a robotic surgical system 300 according to at least one embodiment of the present disclosure. In some examples, the robotic surgical system 300 may implement aspects of or may be implemented by aspects of FIGS. 1, 2A, and 2B as described herein. For example, the robotic surgical system 300 may include a robotic arm 304, which may represent an example of the robotic arm 116 of the robot 114 as described with reference to FIGS. 1, 2A, and 2B. Additionally, the robotic surgical system 300 may include multiple surgical tools 308 (e.g., at least a first surgical tool 308A and a second surgical tool 308B), which may represent examples of the surgical tool 236 as described with reference to FIGS. 2A and 2B. Additionally, while not shown, the robotic surgical system 300 may include a processor, such as the processor 104 as described with reference to FIG. 1, that can assist in controlling and operating the robotic arm 304 and the surgical tools 308.

As described herein and as shown in the example of FIG. 3A, to preempt individual controls and designs for each of the multiple surgical tools 308 needed for the robotic surgical system 300, electronics and control components may be separated from the surgical tools 308 and placed in a single integrated unit at the tip of the robotic arm 304 (e.g., distal end). For example, the single integrated unit may be an interface unit 312 as shown in the example of FIGS. 3A and 3B. In some examples, the interface unit 312 may represent an example of the end mount flange 228 and/or the tool blocks 232 as described with reference to FIGS. 2A and 2B. Additionally or alternatively, the interface unit 312 may be a different component of the robotic surgical system 300 than an end mount flange 228 and/or a tool block 232.

The interface unit 312 may remain connected to the robotic arm 304, while the surgical tools 308 can be interchangeably coupled to the interface unit 312 and can be easily replaced with each other for performing respective operations of a surgical procedure. For example, the interface unit 312 may be designed to support a wide variety of surgical tools 308 and can adjust an operating method for the robotic surgical system 300 to fit which surgical tool is connected or coupled to the interface unit 312. By using the interface unit 312, the surgical tools 308 can have simpler designs that can also be cheaper to manufacture with minimal electronics (e.g., simplifying a sterilization process).

In some examples, the first surgical tool 308A may correspond to a first set of operations (e.g., a first operating method), and the second surgical tool 308B may correspond to a second set of operations (e.g., a second operating method). Accordingly, depending on which surgical tool 308 is coupled to the interface unit 312, the robotic surgical system 300 may perform operations specific to the coupled surgical tool 308. While the first surgical tool 308A and the second surgical tool 308B are shown as specific tools in the example of FIGS. 3A and 3B (e.g., the first surgical tool 308A is shown as an example of a drilling tool, the second surgical tool 308B is shown as an example of a cutting tool or a blade), it is to be understood that the first surgical tool 308A and the second surgical tool 308B may be any type of surgical tool that is designed to be used as part of the robotic surgical system 300.

To determine which surgical tool 308 is attached to the interface unit 312 and coupled to the robotic arm 304 (e.g., in order to determine which operations to perform based on which surgical tool 308 is attached), each surgical tool 308 may include an identification feature that can be used to identify the surgical tool 308 (e.g., scanned, read, accessed, etc.). For example, the identification feature may include a QR code, an S/N, a barcode, an inner memory stored inside each surgical tool 308, a visual marker (e.g., a data Matrix, a shot code, a color code, or any other visual marker not explicitly listed herein), an RFID system (e.g., a radio transponder, a radio receiver, and a radio transmitter disposed on the surgical tools 308, the interface unit 312, the robotic arm 304, etc.), an NFC connection, inner switching of an I/O system of the surgical tool 308, or a combination thereof. Accordingly, after a surgical tool 308 is attached to the interface unit 312, a device of the robotic surgical system 300 may identify the surgical tool 308 based on the corresponding identification feature. For example, a processor may identify which surgical tool 308 is attached to the interface unit 312 (e.g., by accessing the inner memory stored inside the surgical tool 308 after the surgical tool 308 is coupled to the robotic arm 304, by using the RFID system, by using the NFC connection, by using the inner switching of the I/O system, etc.), a camera or imaging device may identify the surgical tool 308 (e.g., by scanning or reading a QR code, S/N, barcode, visual marker, etc. corresponding to the surgical tool 308), or another device not explicitly listed herein may identify the surgical tool 308 based on the identification feature.

Subsequently, after the surgical tool 308 is identified, a processor may determine operations the surgical tool 308 is capable of performing (e.g., by accessing a memory 106, a database 130, and/or a cloud 134 as described with reference to FIG. 1 that stores information associated with each of the surgical tools 308) and may transmit instructions for the surgical tool 308 to perform one of those determined operations (e.g., as part of a surgical procedure). In some examples, the determined operations may be specific to the surgical tool 308 that is attached to the interface unit 312 (e.g., performing incisions, operating a drill, performing a screwing operation, etc.), generic operations for moving and/or actuating the surgical tool 308 (e.g., common operations across multiple surgical tools, such as moving the surgical tool 308 proximate to a target of interest for the surgical procedure), or a combination thereof.

As shown in the example of FIG. 3B, the interface unit 312 may include a set of components 316 configured to provide the coupling between the surgical tools 308 and the robotic arm 304. In some examples, the interface unit 312 may include a body (e.g., storing any elements needed to provide the coupling), a tool receiving element (e.g., for the surgical tool 308 to attach to the interface unit 312), the set of components 316, or a combination thereof.

Additionally, the surgical tools 308 (e.g., the first surgical tool 308A as shown in the example of FIG. 3B) may include a complementary set of components 320 configured to couple the surgical tools 308 to the interface unit 312. For example, the set of components 320 of the surgical tools 308 may be configured to attach to or otherwise couple to the set of components 316 of the interface unit 312. As described herein, the set of components 316 of the interface unit 312 and the set of components 320 of the surgical tools 308 may be configured to provide electrical coupling, communication coupling, mechanical coupling, pneumatic coupling, irrigation coupling, or a combination thereof, for coupling the surgical tools 308 to the robotic arm 304, such that the surgical tools 308 can be operated by a device (e.g., a processor 104 and/or a robot 114 as described with reference to FIG. 1 or another device not explicitly described or listed herein) after being attached to the robotic arm via the interface unit 312 and the corresponding sets of components.

The set of components 316 and the set of components 320 may be one of different types of components or connectors that are configured to provide the coupling between the interface unit 312/robotic arm 304 and the surgical tools 308. For example, the sets of components 316 and 320 may be or may include respective plugs and jacks, pins and holes, wireless communications components, and/or other types of connectors (e.g., threaded connectors, bayonet connectors, push-pull connectors, hose or tube connectors, etc.) that are capable of providing the communication, electrical, mechanical, pneumatic, and irrigation coupling to the surgical tools 308. Additionally, the sets of components 316 and 320 may be connected or coupled together via different mechanisms, such as magnetically connected, wirelessly connected, physically inserted into one another, etc.

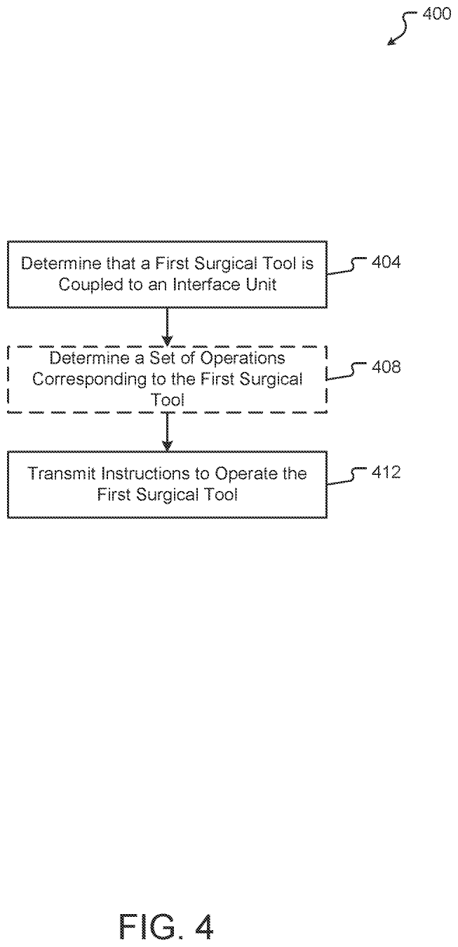

FIG. 4 depicts a method 400 that may be used, for example, to operate a surgical tool that has been coupled to a robotic arm via an interface unit as described herein.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 200 by executing elements stored in a memory such as the memory 106. The elements stored in the memory and executed by the processor may cause the processor to execute one or more steps of a function as shown in method 400. One or more portions of a method 400 may be performed by the processor executing any of the contents of memory, such as an image processing 120, a segmentation 122, a transformation 124, and/or a registration 128.

The method 400 comprises determining that a first surgical tool of a plurality of surgical tools is coupled to an interface unit via a set of components (step 404). For example, the interface unit may be disposed at a distal end of a robotic arm and may be coupled to the robotic arm. Additionally, the interface unit may comprise the set of components, where the set of components are configured to interchangeably couple any surgical tool of the plurality of surgical tools to the robotic arm. For example, the set of components of the interface unit may be configured to provide electrical coupling, communication coupling, mechanical coupling, pneumatic coupling, irrigation coupling, or a combination thereof, for coupling any surgical tool of the plurality of surgical tools to the robotic arm. As described herein, the plurality of surgical tools may include any type of surgical tool that is designed to be used as part of a robotic surgical system as described herein.

In some examples, each surgical tool of the plurality of surgical tools may include an identification feature. Accordingly, the first surgical tool may be determined to be coupled to the interface unit based on identifying the first surgical tool via a first identification feature of the first surgical tool when the first surgical tool is coupled to the interface unit. In some examples, the identification feature may comprise a QR code, an S/N, a barcode, an inner memory stored inside each surgical tool, a visual marker (e.g., a data Matrix, a shot code, a color code, or any other visual marker not explicitly listed herein), an RFID system, an NFC connection, inner switching of an I/O system, or a combination thereof.

In some examples, the method 400 may comprise determining a first set of operations corresponding to the first surgical tool based in part on determining that the first surgical tool is coupled to the interface unit (step 408). For example, the first set of operations may be specific to the first surgical tool, common across the plurality of surgical tools, or a combination thereof. Additionally, the first set of operations may be determined based on identifying the first surgical tool as described previously (e.g., via the first identification feature).

The method 400 also comprises transmitting instructions to operate the first surgical tool based in part on determining that the first surgical tool is coupled to the interface unit (step 412). In some examples, the instructions to operate the first surgical tool may be based on the first set of operations determined previously (e.g., at step 408). Accordingly, the first surgical tool may be operated (e.g., in conjunction with the robotic arm) to perform one or more operations of a surgical procedure (e.g., based on the first set of operations supported by the first surgical tool).

The present disclosure encompasses embodiments of the method 400 that comprise more or fewer steps than those described above, and/or one or more steps that are different than the steps described above.

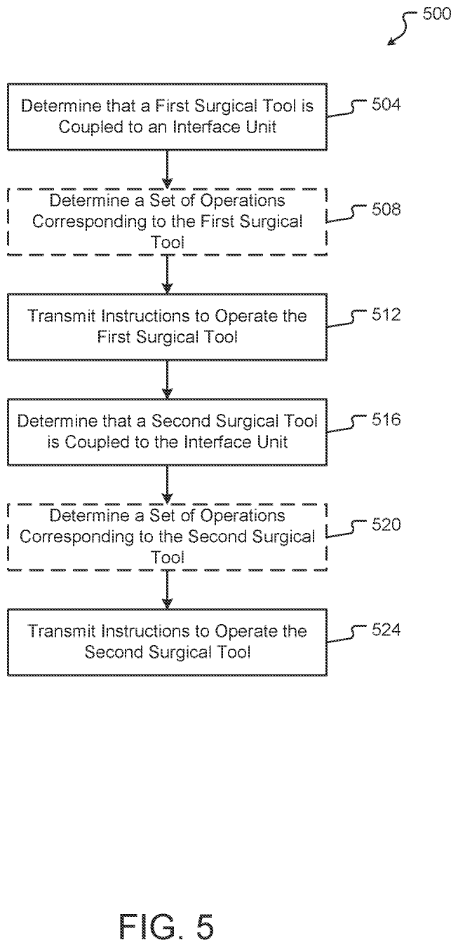

FIG. 5 depicts a method 500 that may be used, for example, to operate an additional surgical tool that has been coupled to a robotic arm via an interface unit as described herein, where the additional surgical tool has replaced a first surgical tool previously coupled to the robotic arm.

The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing elements stored in a memory such as the memory 106. The elements stored in memory and executed by the processor may cause the processor to execute one or more steps of a function as shown in method 500. One or more portions of a method 500 may be performed by the processor executing any of the contents of memory, such as an image processing 120, a segmentation 122, a transformation 124, and/or a registration 128.

In some examples, the method 500 may include steps previously described in greater detail with reference to FIG. 4. For example, the method 500 comprises determining that a first surgical tool of a plurality of surgical tools is coupled to an interface unit via a set of components (step 504). The method 500 also comprises determining a first set of operations corresponding to the first surgical tool based in part on determining that the first surgical tool is coupled to the interface unit (step 508). The method 500 also comprises transmitting instructions to operate the first surgical tool based on determining that the first surgical tool is coupled to the interface unit (step 512). Steps 504, 508, and 512 may correspond to steps 404, 408, and 412, respectively, as described with reference to FIG. 4.

The method 500 also comprises determining that a second surgical tool of the plurality of surgical tools is coupled to the interface unit via the set of components (step 516). In some examples, the second surgical tool may replace the first surgical tool being coupled to the interface unit via the set of components. As described previously, each surgical tool of the plurality of surgical tools may include an identification feature (e.g., a QR code, an S/N, a barcode, an inner memory stored inside each surgical tool, a visual marker, an RFID system, an NFC connection, inner switching of an I/O system, or a combination thereof), where the second surgical tool can be determined to be coupled to the interface unit based on a second identification feature corresponding to the second surgical tool.

In some examples, the method 500 may also comprise determining a second set of operations corresponding to the second surgical tool based in part on determining that the second surgical tool is coupled to the interface unit (step 520). In some examples, the second set of operations may be different than the first set of operations (e.g., determined for the first surgical tool). For example, the second set of operations may be specific to the second surgical tool, common across the plurality of surgical tools, or a combination thereof.

The method 500 also comprises transmitting instructions to operate the second surgical tool based in part on determining that the second surgical tool is coupled to the interface unit (step 524). In some examples, the instructions to operate the second surgical tool may be based in part on the second set of operations. Accordingly, the second surgical tool may be operated (e.g., in conjunction with the robotic arm) to perform one or more operations of a surgical procedure (e.g., based on the second set of operations supported by the second surgical tool).

The present disclosure encompasses embodiments of the method 500 that comprise more or fewer steps than those described above, and/or one or more steps that are different than the steps described above.

As noted above, the present disclosure encompasses methods with fewer than all of the steps identified in FIGS. 4 and 5 (and the corresponding description of the methods 400 and 500), as well as methods that include additional steps beyond those identified in FIGS. 4 and 5 (and the corresponding description of the methods 400 and 500). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges, or steps to those claimed, whether or not such alternate, interchangeable and/or equivalent structures, functions, ranges or steps are disclosed herein, and without intending to publicly dedicate any patentable subject matter.

What is claimed is:

1. A robotic surgical system, comprising:
a robotic arm comprising a proximal end and a distal end;
a plurality of surgical tools;
an interface unit disposed at the distal end of the robotic arm and coupled to the robotic arm, wherein the interface unit comprises a set of components configured to interchangeably couple any surgical tool of the plurality of surgical tools to the robotic arm;
a processor coupled with the robotic arm and the interface unit; and
a memory coupled with and readable by the processor and storing data for processing by the processor, the data, when processed, causes the processor to:
determine that a first surgical tool of the plurality of surgical tools is coupled to the interface unit via the set of components; and
transmit instructions to operate the first surgical tool based at least in part on determining that the first surgical tool is coupled to the interface unit.

2. The robotic system of claim 1, wherein the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm.

3. The robotic surgical system of claim 1, wherein the memory stores further data for processing by the processor that, when processed, causes the processor to:
determine a first set of operations corresponding to the first surgical tool based at least in part on determining that the first surgical tool is coupled to the interface unit, wherein the instructions to operate the first surgical tool are based at least in part on the first set of operations.

4. The robotic surgical system of claim 3, wherein the memory stores further data for processing by the processor that, when processed, causes the processor to:
determine that a second surgical tool of the plurality of surgical tools is coupled to the interface unit via the set of components; and
transmit instructions to operate the second surgical tool based at least in part on determining that the second surgical tool is coupled to the interface unit.

5. The robotic surgical system of claim 4, wherein the memory stores further data for processing by the processor that, when processed, causes the processor to:
determine a second set of operations corresponding to the second surgical tool based at least in part on determining that the second surgical tool is coupled to the interface unit, wherein the instructions to operate the second surgical tool are based at least in part on the second set of operations.

6. The robotic surgical system of claim 5, wherein the second set of operations is different than the first set of operations.

7. The robotic surgical system of claim 5, wherein the second surgical tool replaces the first surgical tool being coupled to the interface unit via the set of components.

8. The robotic surgical system of claim 1, wherein each surgical tool of the plurality of surgical tools comprises an identification feature.

9. The robotic surgical system of claim 8, wherein the data stored by the memory that, when processed, causes the processor to determine the first surgical tool of the plurality of surgical tools is coupled to the interface unit via the set of components causes the processor to:
identify, when the first surgical tool is coupled to the interface unit, the first surgical tool based at least in part on a first identification feature of the first surgical tool.

10. The robotic surgical system of claim 8, wherein the identification feature comprises a quick response (QR) code, a serial number, a barcode, an inner memory stored inside each surgical tool, a visual marker, a radio-frequency identification (RFID) system, a Near-Field Communication (NFC) connection, inner switching of an input/output (I/O) system, or a combination thereof.

11. A robotic surgical system, comprising:
a robotic arm comprising a proximal end and a distal end;
a plurality of surgical tools;
an interface unit disposed at the distal end of the robotic arm and coupled to the robotic arm, wherein the interface unit comprises a set of components configured to interchangeably couple any surgical tool of the plurality of surgical tools to the robotic arm.

12. The robotic surgical system of claim 11, wherein the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm.

13. The robotic surgical system of claim 11, wherein each surgical tool of the plurality of surgical tool corresponds to a specific set of operations.

14. The robotic surgical system of claim 11, wherein each surgical tool of the plurality of surgical tools comprises an identification feature.

15. The robotic surgical system of claim 14, wherein the identification feature comprises a quick response (QR) code, a serial number, a barcode, an inner memory stored inside each surgical tool, a visual marker, a radio-frequency identification (RFID) system, a Near-Field Communication (NFC) connection, inner switching of an input/output (I/O) system, or a combination thereof.

16. A robotic surgical system, comprising:
a robotic arm comprising a proximal end and a distal end;
a plurality of surgical tools;
an interface unit disposed at the distal end of the robotic arm and coupled to the robotic arm, comprising:
a body;
a tool receiving element; and
a set of components configured to interchangeably couple any surgical tool of the plurality of surgical tools to the robotic arm.

17. The robotic surgical system of claim 16, wherein the set of components of the interface unit is configured to provide electrical coupling, communication coupling, mechanical coupling, pneumatic coupling, irrigation coupling, or a combination thereof, for coupling any surgical tool of the plurality of surgical tools to the robotic arm.

18. The robotic surgical system of claim 16, wherein each surgical tool of the plurality of surgical tool corresponds to a specific set of operations.

19. The robotic surgical system of claim 16, wherein each surgical tool of the plurality of surgical tools comprises an identification feature.

20. The robotic surgical system of claim 19, wherein the identification feature comprises a quick response (QR) code, a serial number, a barcode, an inner memory stored inside each surgical tool, a visual marker, a radio-frequency identification (RFID) system, a Near-Field Communication (NFC) connection, inner switching of an input/output (I/O) system, or a combination thereof.
